(19) United States
(12) Patent Application Publication
(10) Pub. No.: US 2025/0359802 A1
(43) Pub. Date: Nov. 27, 2025

(54) METHODS AND APPARATUS TO CALIBRATE ELECTROCARDIOGRAM SYSTEMS

(71) Applicant: Texas Instruments Incorporated, Dallas, TX (US)

(72) Inventors: Aravind Miriyala, Bengaluru (IN); Aatish Chandak, Bengaluru (IN); Akhil C. Sunny, Bangalore (IN); Basavaraj Burge, Bengaluru (IN); Sachin Aithal, Bangalore (IN); Raja Reddy Patukuri, Bengaluru (IN); Sandeep Kesrimal Oswal, Bangalore (IN)

(21) Appl. No.: 18/932,331

(22) Filed: Oct. 30, 2024

(30) Foreign Application Priority Data
May 24, 2024 (IN) 202441040408

(51) Int. Cl. A61B 5/308; A61B 5/00; A61B 5/304

(52) U.S. Cl. CPC A61B 5/308; A61B 5/304; A61B 5/7217; A61B 5/7228; A61B 5/725; A61B 2560/0223

(57) ABSTRACT

An example apparatus includes: an adjustable capacitor having a first terminal coupled to an input terminal, a second terminal coupled to ground, and a control terminal; demodulation circuitry having an input coupled to the first terminal of the adjustable capacitor; calibration circuitry having an input coupled to an output of the demodulation circuitry and an output coupled to the control terminal of the adjustable capacitor; and driver circuitry having an input coupled to the adjustable capacitor and an output coupled to an output terminal.

### CROSS-REFERENCE TO RELATED APPLICATION

This patent application claims the benefit of and priority to Indian Provisional Patent Application No. 202441040408 filed May 24, 2024, which Application is hereby incorporated herein by reference in its entirety.

### TECHNICAL FIELD

This description relates generally to electrocardiogram (ECG) systems and, more particularly, to methods and apparatus to calibrate electrocardiogram systems.

### BACKGROUND

ECG systems are generally used to measure the performance of a human organ, e.g., the heart. Such systems include electrodes that are affixed, e.g., using a patch that sticks to human skin, on various positions across the patient. An ECG device sets a common mode signal of a patient using a first electrode and measures signals produced by the organ using a second and a third electrode. The differences between the signal received at the second electrode and the signal received at the third electrode are indicative of a condition of the organ being measured. For example, an ECG system measuring the performance of a heart may identify conditions including, but not limited to, cardiac rhythm disturbances, inadequate blood flow to certain areas of the heart, electrolyte disturbances, etc.

### SUMMARY

For methods and apparatus to calibrate electrocardiogram systems, an example integrated circuit comprises: an adjustable capacitor having a first terminal coupled to an input terminal of the integrated circuit, a second terminal coupled to ground, and a control terminal; demodulation circuitry having an input coupled to the first terminal of the adjustable capacitor; calibration circuitry having an input coupled to an output of the demodulation circuitry and an output coupled to the control terminal of the adjustable capacitor; and driver circuitry having an input coupled to the adjustable capacitor and an output coupled to an output terminal of the integrated circuit.

An example apparatus comprises a first electrode, a second electrode, and a third electrode, first Low Pass Filter (LPF) circuitry having an input coupled to the first electrode and an output, second LPF circuitry having an input coupled to the second electrode and an output; and front-end circuitry having: a first input terminal coupled to the output of the first LPF circuitry, a first adjustable capacitor coupled to the first input terminal, a second input terminal coupled to the output of the second LPF circuitry, a first output terminal coupled to the third electrode, and a second output terminal coupled to controller circuitry.

An example method comprises: transmitting an excitation signal at an output terminal of front-end circuitry; determining an amplitude and phase of a first input signal received at a first input terminal of the front-end circuitry, wherein the first input signal corresponds to first low pass filter (LPF) circuitry; determining an amplitude and phase of a second input signal received at a second input terminal of the front-end circuitry, wherein the second input signal corresponds to a second LPF circuitry; and matching the phase of the second input signal to the phase of the first input signal by changing, responsive to a difference between the amplitudes, a capacitance of an adjustable capacitor coupled to the second input terminal within the front-end circuitry.

### BRIEF DESCRIPTION OF THE DRAWINGS

FIG. 1 is a block diagram of an example ECG system.

FIG. 2 is a block diagram of an example implementation of the Low Pass Filter (LPF) circuitry of FIG. 1.

FIG. 3 is an illustrative graph of the performance of the front-end circuitry of FIG. 1.

FIG. 4 is a block diagram of an example implementation of the FE circuitry of FIG. 1.

FIG. 5 is a block diagram of an example implementation of the driver circuitry and demodulation circuitry of FIG. 4.

FIG. 6 is an illustrative graph of operations performed by the calibration circuitry of FIG. 4.

FIG. 7 is a flowchart representative of example machine-readable instructions or example operations that may be at least one of executed, instantiated, or performed using one or more of the front-end circuitry and the controller circuitry of FIG. 1 to implement calibration mode.

FIG. 8 is a flowchart representative of example machine-readable instructions or example operations that may be at least one of executed, instantiated, or performed to match phases of input signals as described in FIG. 7.

FIG. 9 is a block diagram of an example processing platform including programmable circuitry structured to execute, instantiate, or perform the example machine-readable instructions or perform the example operations of FIGS. 7 and 8 to implement the system board 100 and front-end circuitry 104 of FIGS. 1, 4, and 5.

The drawings are not necessarily to scale. Generally, the same reference numbers in the drawing(s) and this description refer to the same or similar (functionally and/or structurally) features and/or parts. Although the drawings show regions with clean lines and boundaries, some or all of these lines and boundaries may be idealized. In reality, the boundaries or lines may be unobservable, blended or irregular.

### DETAILED DESCRIPTION

One metric used to evaluate the performance of ECG systems is the Common Mode Rejection Ratio (CMRR). During measurement of an organ, the human body can function like an antenna and absorb various common mode signals. Such signals include both the common mode signal from a transmitting electrode and other extraneous common mode signals in the environment, e.g., 50 Hz or 60 Hz utility frequencies used in various power grid systems. If such common mode signal absorption were left unaddressed, ECG systems would be unable to determine whether the differences between signals received by electrodes were due to a particular property of the organ being measured, due to one or more common mode signals, or due to some combination of the foregoing sources. Accordingly, CMRR quantifies the ability for an ECG system to reject common mode signals before analyzing the difference between signals received by electrodes. In general, a comparatively large CMRR value shows that an ECG system rejects more common mode signals, and is therefore more accurate, than an ECG system with a comparatively small CMRR value.

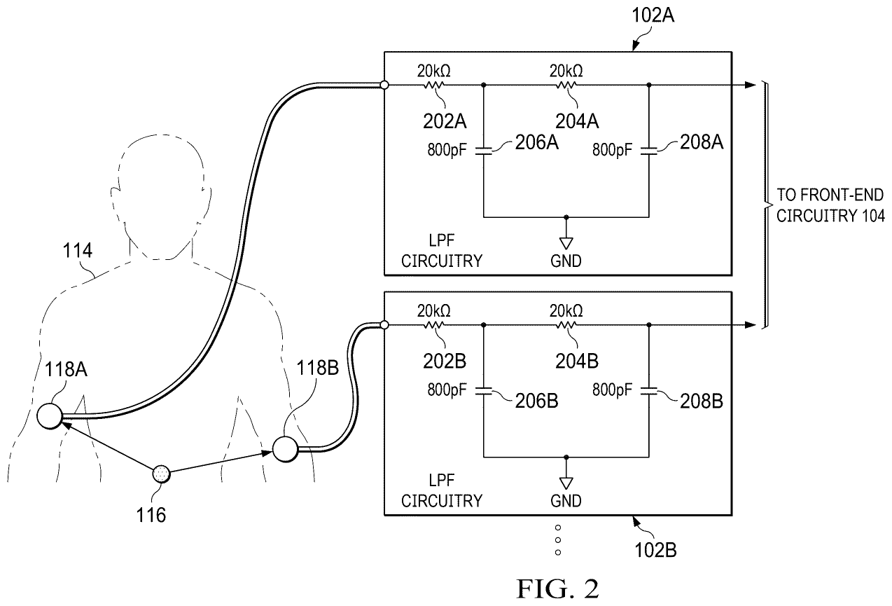

Many ECG systems implement Low Pass Filter (LPF) circuits between the electrodes that receive signals and the device that interprets said signals. In general, LPF circuits benefit an ECG system by rejecting electro-magnetic interference from the environment and performing anti-aliasing operations. However, the amount of frequency removal performed by an LPF circuit is dependent on the values of the electrical components that compose the circuit. If values of the electrical components of a first LPF circuit do not match the values of the electrical components in a second LPF, the two circuits perform a different amount of filtering. Such component mismatch between LPF circuits degrades CMRR because the component mismatch results in a difference between electrode signals received at the analysis device that is not due to the performance of the organ being measured. LPF circuits and component mismatch are described further in connection with FIG. 2.

Many electrical components are marketed with a tolerance value that reflects the precision of the manufacturing process of that component. For example, a 100 Ohm (Ω) resistor that is marketed with a 1.0% tolerance has an actual resistance anywhere between [99 Ω, 101Ω]. In general, the cost of an electrical component increases as the marketed tolerance value decreases. For example, resistors marketed with a 0.1% tolerance are approximately 1.5 times more expensive than resistors marketed with 1.0% tolerance. Also, capacitors marketed with 1.0% tolerance are approximately 5 times more expensive than capacitors marketed with 5.0% tolerance. Accordingly, manufacturers and designers cannot practically increase the CMRR of ECG systems using components with tighter tolerances because such an approach is not cost effective. Moreover, the designer or manufacturer of the LPF circuits used in an ECG system is often a different and independent entity from the designer or manufacturer of the electrode analysis device. Thus, the designer or manufacturer of such an electrode analysis device may be unable to determine or rely on the tolerances or amount of component mismatch within the LPF circuits.

Some electrode analysis devices attempt to improve CMRR by measuring the amount of component mismatch in the LPF circuits and by then using the measurement to account for the mismatch when utilizing digital signal processing to process the signals from the electrodes. However, such a measurement requires single-ended processing of the signals that are received by the LPF circuits, which requires additional analog components such as extra amplifier circuits and extra Analog-to-Digital Converter (ADC) circuits when compared to other electrode analysis devices that use differential-ended processing.

The extra amplifier circuits and extra ADC circuits that process single-ended signals also must be rated to support higher voltages than comparable circuits that process differential-ended signals. For example, suppose two electrode signals have 1.05 Volts peak to peak (Vpp) and 1.00 Vpp, respectively. The extra amplifier circuits and extra ADC circuits used in single-ended processing, e.g., the circuits that support digital corrections to component mismatch, must be rated to have at least 1.05 Vpp applied across the components to process the proposed electrode signals. In contrast, the amplifier circuits and ADC circuits used in differential processing only need to be rated to have (1.05-1.00)=50 mVpp applied across the components to process the proposed electrode signals.

Performance of the digital signal processing operations that account for component mismatch require greater processing speeds than other electrode analysis devices. For example, a device that performs digital signal processing to account for component mismatch requires a clock signal with a period of approximately 40 nanoseconds (ns). In contrast, an electrode analysis device that does not perform digital signal processing to account for component mismatch only requires a clock signal with a period of approximately 250 ns. The additional hardware components, the higher voltage rating required for such components, and the faster processing speed required to digitally account for component mismatch, collectively add significant cost, area, power consumption, and complexity to electrode analysis devices that implement such a technique.

Example methods, apparatus, and systems described herein improve CMRR in ECG systems using a simple, low-power, and cost-effective manner. An example electrode analysis device corrects for component mismatch in LPF circuits in the analog domain rather than the digital domain. To do so, the example electrode analysis device supports a calibration mode, during which pre-existing driver circuitry is used to transmit an excitation signal through the transmission electrode. The excitation signal is received by the other electrodes and passes through all of the LPF circuits. The difference between the phase of the output signals generated by the LPF circuits is responsive to the amount of component mismatch between the LPF circuits. Accordingly, pre-existing phase detection circuitry is used during calibration mode to detect the phase of the individual signals. Example detection circuitry then adjusts one or more adjustable capacitors so that the phase of the incoming signals match one another, thereby correcting for the component mismatch in the analog domain. Such an approach requires fewer components, lower voltage ratings, and lower processing speeds than electrode analysis devices that account for component mismatch in the digital domain.

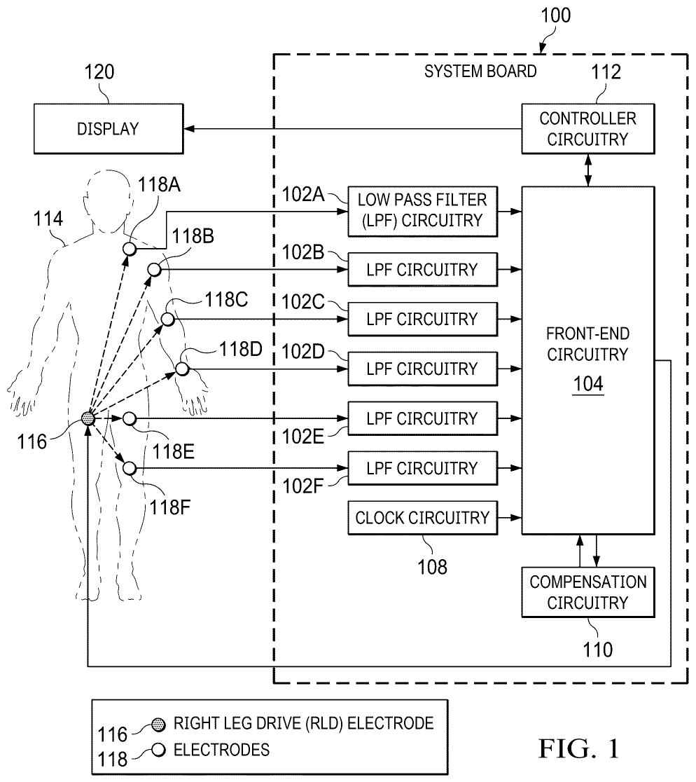

FIG. 1 is a block diagram of an example ECG system. The example ECG system of FIG. 1 includes a system board 100. The system board 100 includes Low Pass Filter (LPF) circuitry 102A, 102B, . . . , 102F (collectively referred to as LPF circuits 102), front-end circuitry 104, clock circuitry 108, compensation circuitry 110, and controller circuitry 112. The example of FIG. 1 also includes a patient 114, a Right Leg Drive (RLD) electrode 116, electrodes 118A, 118B, . . . , 118F (collectively referred to as electrodes 118), and a display 120.

Within the system board 100, the LPF circuits 102 remove high frequency components of input signals to reject electro-magnetic interference and perform anti-aliasing as described above. A given LPF circuit 102A has an input coupled to a corresponding electrode 118A and an output coupled to the front-end circuitry 104. The example of FIG. 1 shows six LPF circuits 102A, 102B, . . . , 102F. In other examples, the system board 100 includes any number of LPF circuits 102. The LPF circuits 102 are described further in connection with FIG. 2.

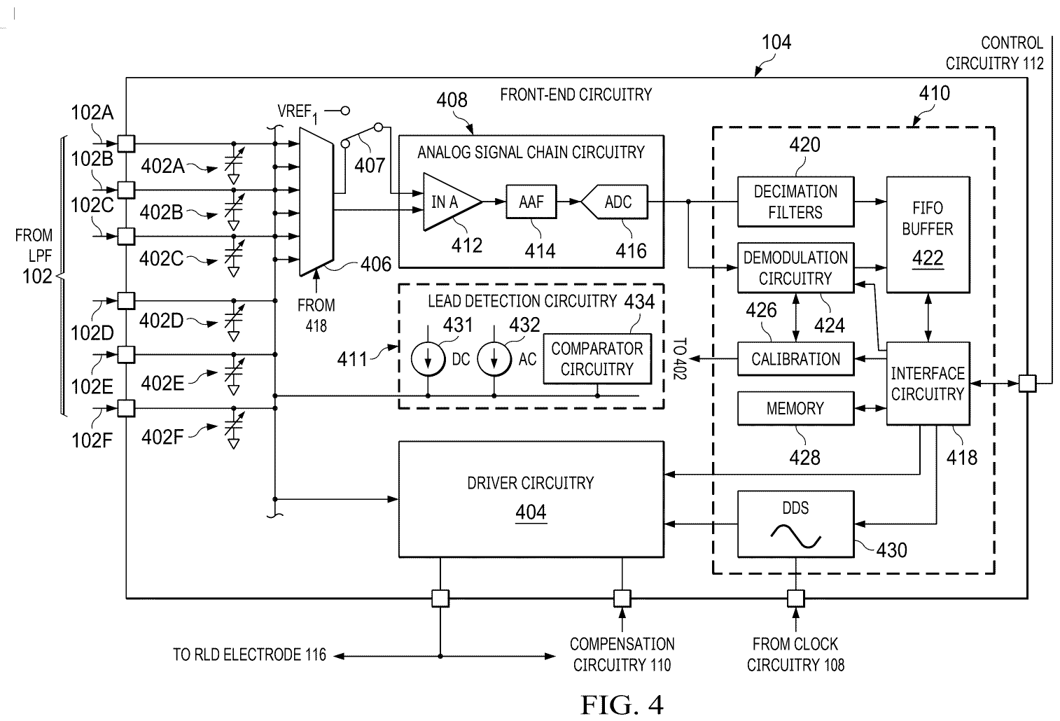

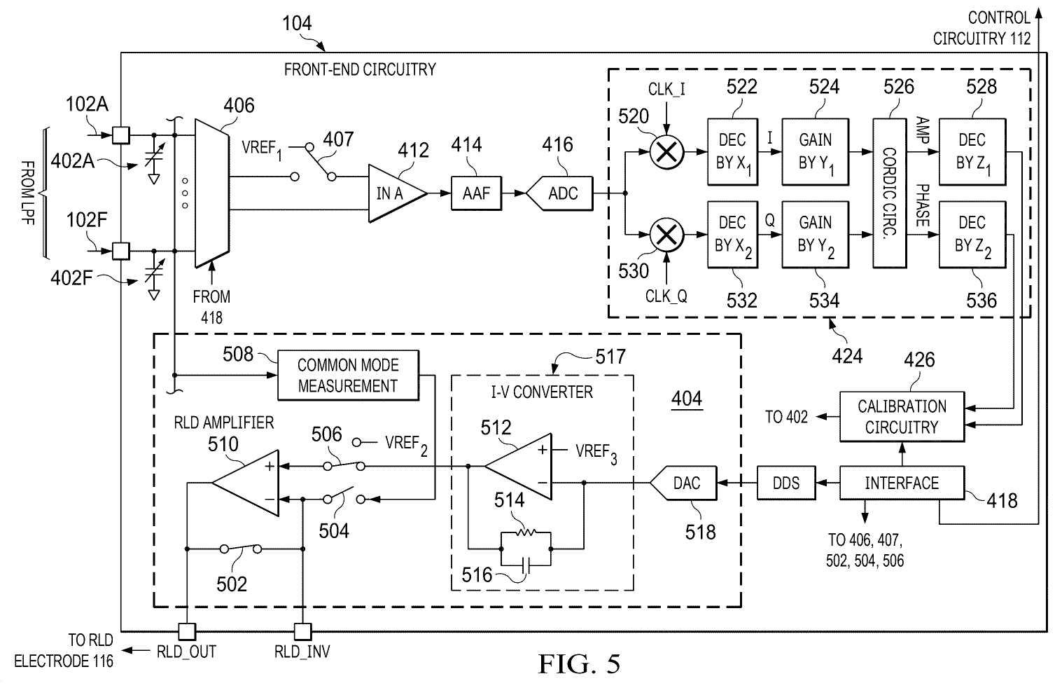

The front-end circuitry 104 has one input per LPF circuit on the system board 100. The front-end circuitry 104 also has an input coupled to the clock circuitry 108, an input coupled to the compensation circuitry, and an input coupled to the controller circuitry. The front-end circuitry 104 also has an output coupled to the compensation circuitry 110, an output coupled to the controller circuitry 112, and an output coupled to the to the RLD electrode 116. The front-end circuitry 104 implements a calibration mode, as described in examples herein, that compensates for component mismatch within the LPF circuits 102. The front-end circuitry 104 also sets a common mode of the human body using the RLD electrode 116 and analyzes the one or more returning signals to measure the performance of an organ within the patient 114. In the example of FIG. 1, the front-end circuitry 104 is designed and manufactured independently of the LPF circuits 102. The front-end circuitry 104 is described further in connection with FIGS. 4 and 5.

The clock circuitry 108 has an output coupled to the front-end circuitry 104. The clock circuitry 108 provides a periodic reference signal, e.g., a clock signal, which is used by the front-end circuitry 104 to perform timing-based operations.

The compensation circuitry 110 has an input and an output that are both coupled to the front-end circuitry 104. The front-end circuitry 104 uses the compensation circuitry 110 when measuring the performance of an organ to stabilize the common mode signal that is transmitted to the RLD electrode 116. As used herein, operations performed by the front-end circuitry 104 while measuring the performance of an organ in the patient 114 is referred to as “measurement mode”.

The controller circuitry 112 has an input and an output that are both coupled to the front-end circuitry 104. The controller circuitry 112 instructs the front-end circuitry 104 when to enter calibration mode. The controller circuitry 112 also receives analysis results from the front-end circuitry 104 and provides the results, e.g., data that represents the performance of the organ being measured, on the display 120. The controller circuitry 112 may be implemented by any type of programmable circuitry. Examples of programmable circuitry include but are not limited to programmable microprocessors, Field Programmable Gate Arrays (FPGAs) that may instantiate instructions, Central Processor Units (CPUs), Graphics Processor Units (GPUs), Digital Signal Processors (DSPs), XPUs, or microcontrollers and integrated circuits such as Application Specific Integrated Circuits (ASICs).

FIG. 2 is a block diagram of an example implementation of the Low Pass Filter (LPF) circuitry of FIG. 1. The example of FIG. 2 includes the LPF circuits 102A and 102B, the patient 114, and the electrodes 118A and 118B. The LPF circuitry 102A includes example resistors 202A, 204A, and example capacitors 206A, 208A. The LPF circuitry 102B includes example resistors 202B, 204B, and example capacitors 206B, 208B.

Within the LPF circuitry 102A, the resistor 202A has a first terminal coupled to the electrode 118A and a second terminal. The resistor 204A has a first terminal coupled to the second terminal of the resistor 202A. The resistor 204A also has a second terminal coupled to the front-end circuitry 104. The capacitor 206A has a first terminal coupled to the second terminal of the resistor 202A and coupled to the first terminal of the resistor 204A. The capacitor 206A also has a second terminal coupled to ground. The capacitor 208A has a first terminal coupled to the second terminal of the resistor 204A and coupled to the front-end circuitry 104. The capacitor 208A also has a second terminal coupled to ground.

The LPF circuitry 102B, and more generally the rest of the LPF circuits 102, have the same components coupled together in the same configuration as the LPF circuits 102A. Accordingly, in theory, a given LPF circuit, e.g., LPF circuitry 102A, theoretically performs the same signal editing operations as any other LPF circuit within the system board 100, e.g., LPF circuitry 102B.

The values 20 kΩ and 800 pF are shown as examples within FIG. 2. More generally, the LPF circuits 102 can be implemented with any resistance or capacitance values. But regardless of the specific values chosen, the resistors 202 across the LPF circuits 102 are designed to have the same resistance value, the resistors 204 across the LPF circuits 102 are designed to have the same resistance value, the capacitors 206 across the LPF circuits 102 are designed to have the same capacitance value, and the capacitors 208 across the LPF circuits 102 are designed to have the same capacitance value. Thus, component mismatch as described above occurs when any of the resistors 202A, 202B, . . . , 202F have different resistance values from one another, when any of the resistors 204A, 204B, . . . , 204F have different values from one another, etc.

During measurement mode, the front-end circuitry 104 measures the difference in signals between two signals received from the electrodes 118A, 118B. Using the LPF architecture shown in the example of FIG. 2, the difference in voltage between the outputs of the LPF circuitry 102A and the LPF circuitry 102B is given by equation (1):

$${V}_{102 A} - {V}_{102 B} = \frac{1}{1 + 3 s ( R - Δ R ) ( C - Δ C ) + {( s ( R - Δ R ) ( C - Δ C ) )}^{2}} - \frac{1}{1 + 3 s ( R + Δ R ) ( C + Δ C ) + {( s ( R + Δ R ) ( C + Δ C ) )}^{2}} \qquad ( 1 )$$

Equation (1) can be simplified and approximated using equation (2):

$${V}_{102 A} - {V}_{102 B} \approx 6 sRC ( \frac{Δ R}{R} + \frac{Δ C}{C} ) \qquad ( 2 )$$

In equations (1) and (2), V_{102A} refers to the voltage at the output of the LPF circuitry 102A and V_{102B} refers to the voltage at the output of the LPF circuitry 102B. s is a complex variable that represents frequency. R refers to the expected resistance value of the resistors 202 and 204, e.g., 20 kΩ. Equations (1) and (2) describes a worst-case scenario for component mismatch due to manufacturing tolerances. In particular, the value of V_{102A}−V_{102B} in equations (1) and (2) is determined presuming: the resistors 204A and 204B have a value of R−ΔR, the capacitors 206A and 208A have a value of C−ΔC, the resistors 202B and 204B have a value of R+ΔR, and the capacitors 206B and 208B have a value of C+ΔC.

Equation (2) shows that the differential voltage analyzed by the front-end circuitry 104, (V_{102A}−V_{102B}), is directly proportional to the component mismatch ΔR and ΔC. Thus, an increase in component mismatch of two or more LPF circuits 102 directly decreases the CMRR of the system board 100.

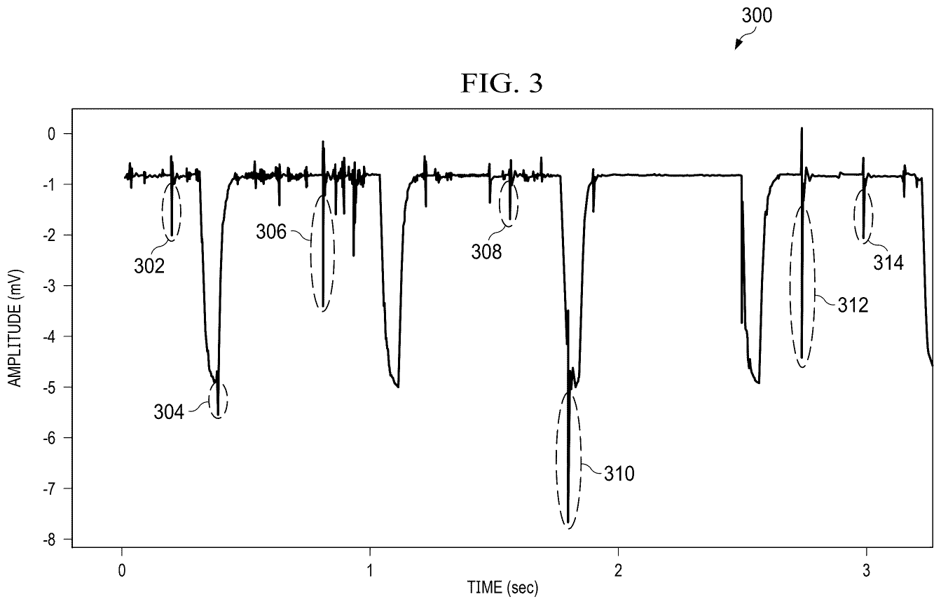

FIG. 3 is an illustrative graph of the performance of the front-end circuitry of FIG. 1. The example graph of FIG. 3 shows a signal 300 that has a regions 302, 304, 306, 308, 310, 312, 314. The signal 300 represents how the value of (V_{102A}−V_{102B}) from FIG. 2 changes over time in the presence of electro-surgical interference (ESI). ESI refers to common mode interference due to electro-surgery, a type of surgery performed in using as an electrical knife to cut tissue, destroy tissue, or stop internal bleeding within the patient 114.

In general, the signal 300 represents a signal produced by an organ (e.g., a heart). The signal 300 shows that the value of (V_{102A}−V_{102B}) generally rests at approximately −1 mV. The signal 300 also includes multiple local minima that have various peak amplitudes. The front-end circuitry 104 interprets the local minima as a signal produced by an organ. However, the local minima also include the regions 302, 304, 306, 308, 310, 312, 314, which occur due to poor rejection of ESI common mode signal caused by component mismatch within the LPF circuits 102A and 102B. The front-end circuitry 104 may be unable to correctly interpret such regions because the circuit has no information to distinguish which part of the peak is due to the organ and which part is due to poor rejection of ESI caused by component mismatch. More generally, component mismatch limits the performance of ECG systems because the front-end circuitry 104 cannot determine which part of the differential signal is attributable to the performance of the organ being measured and which part is due to poor CMRR caused by component mismatch.

FIG. 4 is a block diagram of an example implementation of the front-end circuitry of FIG. 1. In the example of FIG. 4, the front-end circuitry 104 includes adjustable capacitors 402A, 402B, . . . , 402F (collectively referred to as adjustable capacitors 402), driver circuitry 404, multiplexer circuitry (mux) 406, a switch 407, analog signal chain circuitry 408, digital circuitry 410, and lead detection circuitry 411. The analog signal chain circuitry 408 includes instrumentation amplifier (INA) circuitry 412, anti-aliasing filter (AAF) circuitry 414, and analog to digital conversion (ADC) circuitry 416. The digital circuitry 410 includes decimation filters 420, interface circuitry 418, a first-in-first-out (FIFO) buffer 422, demodulation circuitry 424, calibration circuitry 426, memory 428, and direct digital synthesis (DDS) circuitry 430. The lead detection circuitry 411 include a DC current source 431, an ΔC current source 432, and comparator circuitry 434.

The adjustable capacitor 402A has a first terminal coupled to the output of the LPF circuitry 102A, the adjustable capacitor 402B has a first terminal coupled to the output of the LPF circuitry 102B, . . . , and the adjustable capacitor 402F has a first terminal coupled to the output of the LPF circuitry 102F. The respective adjustable capacitors 402 also have second terminals that are coupled to ground and control terminals that are coupled to the calibration circuitry 426.

The driver circuitry 404 has a first input coupled to the first terminals of the adjustable capacitors 402. The driver circuitry 404 has a second input coupled to the compensation circuitry 110. The driver circuitry 404 also has an output that is coupled to both the compensation circuitry 110 and the RLD electrode 116. During measurement mode, the driver circuitry 404 generates and transmits a common mode signal to the RLD electrode 116. The driver circuitry 404 also performs different operations in calibration mode to transmit an excitation signal to the RLD electrode 116. The driver circuitry 404 is described further in connection with FIG. 5.

The mux 406 has an input coupled to the LPF circuitry 102A and the first terminal of the adjustable capacitor 402A, an input coupled to the LPF circuitry 102B and the first terminal of the adjustable capacitor 402B, . . . , and an input coupled to the LPF circuitry 102F and the first terminal of the adjustable capacitor 402F. The mux 406 also has a first output coupled to the switch 407 and a second output coupled to the instrumentation amplifier circuitry 412, and a control terminal that is coupled to the interface circuitry 418. The mux 406 passes two signals from its various inputs to its outputs responsive to the voltage received at the control terminal.

The switch 407 includes a first terminal coupled to the output of the mux 406, a second terminal that receives a reference voltage (V_{REF1}), a third terminal coupled to the INA circuitry 412, and a control terminal. The switch 407 provides the input of the INA circuitry 412 with either the VREF1 signal or the signal at the first output of the mux 406. The switch 407 determines which signal to pass to the INA circuitry 412 responsive to the interface circuitry 418.

Within the analog signal chain circuitry 408, the INA circuitry 412 includes first and second inputs coupled to the first and second outputs of the mux 406. the INA circuitry 412 also has an output coupled to the AAF circuitry 414. The INA circuitry 412 computes the difference in voltage between the two signals it receives at its inputs. The INA circuitry 412 also outputs a signal that increases the gain of said difference. The INA circuitry 412 also performs CMRR operations by supporting high input impedance and providing low output impedance.

The AAF circuitry 414 has an input coupled to the output of the INA circuitry 412. The AAF circuitry 414 also has an output coupled to the ADC circuitry 416. The AAF circuitry 414 performs filtering operations to restrict the bandwidth of a signal to satisfy the Nyquist-Shannon sampling theorem over the band of interest.

The ADC circuitry 416 has an input coupled to the output of the AAF circuitry 414. The ADC circuitry 416 also has an output coupled to both the decimation filters 420 and the demodulation circuitry 424. The ADC circuitry 416 converts the analog signal provided by the AAF circuitry 414 into digital values.

In general, the digital circuitry 410 refers to one or more circuits that use digital logic to analyze the values produced by the ADC circuitry 416 and sends control signals to various analog components within the front-end circuitry 104. The digital circuitry 410 of FIG. 4 may be instantiated (e.g., creating an instance of, bring into being for any length of time, materialize, implement, etc.) by programmable circuitry such as a Central Processor Unit (CPU) executing first instructions. Also or alternatively, the digital circuitry 410 of FIG. 4 may be instantiated (e.g., creating an instance of, bring into being for any length of time, materialize, implement, etc.) by (i) an Application Specific Integrated Circuit (ASIC) or (ii) a Field Programmable Gate Array (FPGA) structured or configured in response to execution of second instructions to perform operations corresponding to the first instructions. Some or all of the digital circuitry 410 may, thus, be instantiated at the same or different times. Some or all of the digital circuitry 410 may be instantiated, for example, in one or more threads executing concurrently on hardware or in series on hardware. Moreover, in some examples, some or all of the digital circuitry 410 may be implemented by microprocessor circuitry executing instructions or FPGA circuitry performing operations to implement one or more virtual machines or containers.

Within the digital circuitry 410, the interface circuitry 418 transmits and receives data from the controller circuitry 112. The data may be used for any purpose related to the ECG system. In the example of FIG. 4, the interface circuitry 418 implements the Serial Peripheral Interface (SPI) protocol to support communication with the controller circuitry 112. In other examples, the interface circuitry 418 and the controller circuitry 112 communicate using a different protocol.

The decimation filters 420 process the values produced by the ADC circuitry 416 in the FIFO buffer 422. The demodulation circuitry 424 analyzes the full stream of values produced by ADC circuitry 416 to produce output data. Before the performance of an organ in the patient 114 is measured, the demodulation circuitry 424 reviews the digital values to identify the amplitude of a signal from the AC current source 432 in the lead detection circuitry 411. The demodulation circuitry 424 identifies the AC signal amplitude data to determine whether the RLD electrode 116 and the electrodes 118 are properly connected to the patient 114. The demodulation circuitry 424 then stores AC signal amplitude data in the FIFO buffer 422. The FIFO temporarily stores data until the interface circuitry 418 is ready to transmit said data to the controller circuitry 112.

During calibration mode, the demodulation circuitry 424 is used to measure the amplitude and phase of individual signals corresponding to individual ones of the LPF circuits 102. The demodulation circuitry 424 provides data produced during calibration mode to the calibration circuitry 426 in addition to, or instead of, the FIFO buffer 422. The demodulation circuitry 424 is described further in connection with FIG. 5.

The calibration circuitry 426 has outputs coupled to the control terminals of the adjustable capacitors 402. During calibration mode, the calibration circuitry 426 changes the values of one or more adjustable capacitors responsive to the amplitude and phase measurements performed by the demodulation circuitry 424. Component mismatch in the LPF circuits 102 generally decreases performance of the ECG system of FIG. 1 To prevent issues, the calibration circuitry 426 changes values of the one or more adjustable capacitors so that the input electrode signals all have the same phase, regardless of the location or quantity of component mismatch that exists upstream in the LPF circuits 102. Thus, when the front-end circuitry 104 begins to measure the performance of an organ after calibration mode, the controller circuitry 112 knows the difference in electrode signals is due to the patient 114 and not component mismatch.

The memory 428 stores data used by various components of the digital circuitry 410 to perform operations. For example, the memory 428 may store settings used to by the demodulation circuitry 424 to perform operations. The memory 428 may be implemented as any type of memory. For example, the memory 428 may be a volatile memory or a non-volatile memory. The volatile memory may be implemented by Synchronous Dynamic Random Access Memory (SDRAM), Dynamic Random Access Memory (DRAM), and/or any other type of RAM device. The non-volatile memory may be implemented by flash memory and/or any other desired type of memory device.

The DDS circuitry 430 has an input coupled to the interface circuitry 418 and an output coupled to the driver circuitry 404. The DDS circuitry 430 creates arbitrary waveforms using the signal from the clock circuitry 108 as a reference. The DDS circuitry 430 generates waveforms with specific timing and shape characteristics responsive to instructions that are forwarded from the interface circuitry 418. For example, the DDS circuitry 430 generates a sine wave during calibration mode responsive to the interface circuitry 418. In some examples, the signal generated by the DDS circuitry 430 may be referred to as an excitation signal.

The lead detection circuitry 411 represents analog components used in conjunction with the demodulation circuitry 424 to detect, prior to the measurement of an organ, whether the RLD electrode 116 and the electrodes 118 are attached properly to the patient 114. In general, the lead detection circuitry 411 performs such an analysis by transmitting a test signal generated by one of the DC current source 431 or the AC current source 432, then using the comparator circuitry 434 to comparing the input signal(s) that are received from the LPF circuits 102 with an expected value.

The adjustable capacitors 402, the driver circuitry 404, the demodulation circuitry 424, calibration circuitry 426, and the DDS circuitry 430 collectively operate during calibration mode to provide an analog correction for component mismatch of the LPF circuits 102. Notably, the clock circuitry 108 used by the DDS circuitry 430 and other components of the digital circuitry 410 has a lower frequency than other electrode analysis devices that compensate for component mismatch in the digital domain. Furthermore, while other electrode analysis devices require one copy of the analog signal chain circuitry 408 per LPF circuit to independently and simultaneously digitize the incoming signals, the example of FIG. 4 shows the front-end circuitry 104 requires only one instance of the analog signal chain circuitry 408 per differential signal being generated. Accordingly, the front-end circuitry 104 described in examples herein improves CMRR in a manner that is simpler, consumes less power, can be implemented with less space, and costs less than other electrode analysis devices.

FIG. 5 is a block diagram of an example implementation of the driver circuitry 404 and demodulation circuitry 424 of FIG. 4. The example of FIG. 5 shows that the driver circuitry 404 includes switches 502, 504, and 506, common mode measurement circuitry 508, amplifier circuitry 510 and 512, a resistor 514, a capacitor 516, and digital to analog converter (DAC) circuitry 518. The example of FIG. 5 also shows that the demodulation circuitry 424 includes multiplier circuits 520 and 530, decimation circuits 522 and 532, gain circuitry 524 and 534, coordinate rotation digital computer (CORDIC) circuitry 526, and decimation circuitry 528 and 536. In some examples, the amplifier circuitry 510 is referred to as right leg drive amplifier circuitry 510.

The example of FIG. 5 shows the operations of the front-end circuitry 104 when operating in calibration mode. Thus, components shown in FIG. 4 but not in FIG. 5 are still present within the front-end circuitry 104 but may not be utilized during the calibration mode.

During measurement mode, the driver circuitry 404 provides an initial common mode signal to the RLD electrode 116, then provides adjustments responsive to the incoming signals provided by the electrodes 118 and LPF circuits 102. During such operations, the common mode measurement circuitry 508 measures and provides the actual common mode signal to the negative terminal of the RLD amplifier circuitry 510. The RLD amplifier circuitry 510 then compares the actual common mode signal to the expected common mode signal and transmits an output, at the RLD_OUT terminal, responsive to the comparison. The expected common signal is provided by the system board 100 as a reference voltage (V_{REF2}). During such operations, the compensation circuitry 110 modifies the common mode signal by performing stabilization operations. The compensation circuitry 110 can perform such signal transformation because, while the performance of an organ in the patient 114 is being measured, the compensation circuitry 110 is coupled to both the output terminal and the negative terminal of the RLD amplifier circuitry 510 via the RLD_OUT and RLD_INV terminals.

During the calibration mode, the driver circuitry 404 is used to transmit an excitation signal provided by the DDS circuitry 430. Accordingly, the example of FIG. 5 shows the driver circuitry 404 includes the switches 502, 504, and 506 coupled to the interface circuitry 418. In the example of FIG. 5, the controller circuitry 112 provides instructions, via the interface circuitry 418, that switch the front-end circuitry 104 between measurement mode and calibration mode. In other examples, programmable circuitry within the front-end circuitry 104 sends the instructions between measurement mode and calibration mode.

The switch 502 includes a first terminal coupled to the output terminal of the RLD amplifier circuitry 510, a second terminal coupled to the negative terminal of the RLD amplifier circuitry 510, and a control terminal coupled to the interface circuitry 418. Responsive to instructions from the controller circuitry 112, the switch 502 operates in the open state during measurement mode and operates in the closed state during calibration mode. The closed state in calibration mode creates a short circuit between the output and negative terminals of the RLD amplifier circuitry 510, thereby repurposing the RLD amplifier circuitry 510 to operate as a buffer. The closed state of switch 502 also functionally decouples the compensation circuitry 110 from the front-end circuitry 104 during calibration mode.

The switch 504 includes a first terminal coupled to the negative terminal of the RLD amplifier circuitry 510, a second terminal coupled to the common mode measurement circuitry 508, and a control terminal coupled to the interface circuitry 418. Responsive to instructions from the controller circuitry 112, the switch 504 operates in the closed state during measurement mode and operates in the open state during calibration mode. The open state of switch 504 decouples the common mode measurement circuitry 508 from the RLD amplifier circuitry 510 during calibration mode.

The switch 506 includes a first terminal coupled to the positive terminal of the RLD amplifier circuitry 510, a second terminal that receives an expected common mode signal (labeled V_{REF2} in FIG. 5), a third terminal coupled to the output of the amplifier circuitry 512. Responsive to instructions from the controller circuitry 112, the switch 506 couples to the second terminal to receives V_{REF2} during measurement mode and couples to the amplifier circuitry 512 during calibration mode. The amplifier circuitry has a positive terminal coupled to a reference voltage (V_{REF3}), a negative terminal coupled to the DAC circuitry 518, the resistor 514, and the capacitor 516, and an output terminal coupled to the switch 506, the resistor 514, and the capacitor 516. The amplifier circuitry 512, the resistor 514, and the capacitor 516, collectively form current-to-voltage converter circuitry 517 that prepares the signal produced by the DAC circuitry 518. The DAC circuitry 518, in turn, generates an analog version of the digital excitation signal produced by the DDS circuitry 430. Accordingly, the switch 506 enables the positive terminal of the RLD amplifier circuitry 510 to receive an analog version of the excitation signal during calibration mode.

The electrodes 118 are shorted to one another during calibration mode so that each of the LPF circuits 102 receive the same excitation signal. The mux 406 then passes, responsive to the controller circuitry 112, one input signal at a time to the analog signal chain circuitry 408 for eventual analysis by the demodulation circuitry 424. During calibration mode, the controller circuitry 112 instructs the switch 407 to transition away from the mux 406 and to instead receive V_{REF1}. Accordingly, the differential signal produced by the INA circuitry 412 is responsive to separate input signals during measurement mode but is responsive to only one input signal during calibration mode. The switch 407 therefore enables the demodulation circuitry 424 to measure the amplitude and the phase of the input signals individually and independently from one another.

Within the demodulation circuitry 424, the multiplier circuit 520 has an input coupled to the ADC circuitry 416 and an output coupled to the decimation circuitry 522. The multiplier circuit 520 performs multiplication operations to combine the data stream produced by the ADC circuitry 416 with a clock signal.

The decimation circuitry 522 has an input coupled to the multiplier circuitry 520 and an output coupled to the gain circuitry 524. The decimation circuitry 522 produced an output signal that has fewer data points than the input signal. The proportion of data points removed by the decimation circuitry 522, labeled x₁ in FIG. 5, may be edited by the controller circuitry 112.

The gain circuitry 524 has an input coupled to the decimation circuitry 522 and an output coupled to the CORDIC circuitry 526. The gain circuitry 524 increases the magnitude of the digital values received from the decimation circuitry 522. The difference in magnitude between the input and output of the gain circuitry 524, labeled y₁, may be edited by the controller circuitry 112.

The CORDIC circuitry 526 has an input coupled to the gain circuits 524, 534 and an output coupled to decimation circuitry 528. In general, the CORDIC circuitry 526 implements operations to perform floating-point arithmetic in a time and data-efficient manner.

The decimation circuitry 528 has an input coupled to the CORDIC circuitry 526 and an output coupled the calibration circuitry 426. The decimation circuitry 528 removes a portion of the data points that represent the amplitude measurement. The proportion of data points removed by the decimation circuitry 522, labeled z₁ in FIG. 5, may be edited by the controller circuitry 112.

Within the demodulation circuitry 424, the multiplier circuitry 530, decimation circuitry 532, gain circuitry 534, and decimation circuitry 536 couple to one another and perform operations in the same manner as the multiplier circuitry 520, decimation circuitry 522, gain circuitry 524, and decimation circuitry 528, respectively. However, the CORDIC circuitry 526 uses the output of the gain circuits 524 and 534 to determine the phase of the input signal. Furthermore, the parameters of decimation circuitry 532, gain circuitry 534, and decimation circuitry 536, labeled x₂, y₂, and z₂ in FIG. 5 respectively, may be different values than the decimation circuitry 522, gain circuitry 524, or decimation circuitry 528. During measurement mode, the demodulation circuitry 424 uses a first set of values for x₁, y₁, z₁, x₂, y₂, and z₂ to determine the amplitude of the difference between two input signals that indicate the contact impedance of electrodes to the patient 114. During calibration mode, the controller circuitry 112 changes the values of one or more of x₁, y₁, z₁, x₂, y₂, and z₂ to measure the amplitude and phase of individual input signals that correspond to a previous excitation signal transmission.

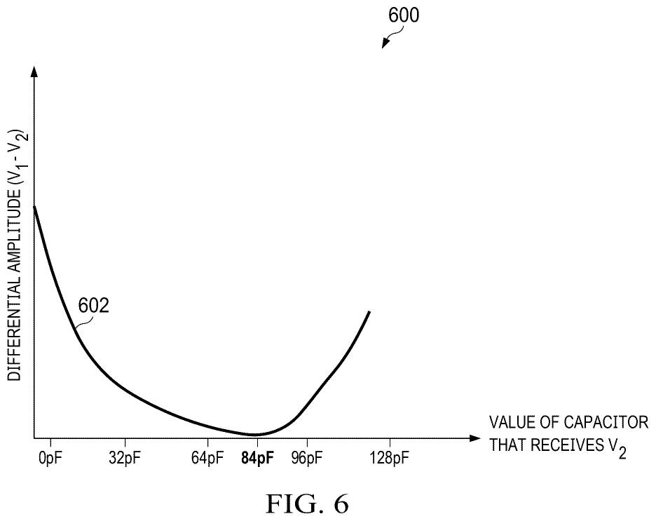

The calibration circuitry 426 includes a first input coupled to the output of the decimation circuitry 528, a second input coupled to the output of the decimation circuitry 536, and multiple outputs coupled to the control terminals of the adjustable capacitors 402. During calibration mode, the calibration circuitry 426 selects one of the input signals received from the LPF circuits 102. The calibration circuitry 426 then adjusts one or more of the adjustable capacitors 402 so that the phases of the other input signals received from the other LPF circuits match the phases of the selected input signal. By setting the phases of the input signals equal during calibration mode, the calibration circuitry 426 ensures that any phase differences observed during a subsequent measurement mode are independent of any component mismatch within the LPF circuits 102. The technique used by the calibration circuitry 426 to change the capacitance of one or more adjustable capacitors 402 is described further in connection with FIGS. 6, 7, and 8.

FIG. 6 is an illustrative graph of operations performed by the calibration circuitry of FIG. 4. FIG. 6 shows an example graph 600 having a signal 602. The y axis of the graph 600 shows the difference, in Volts, between V₁ and V₂, where V₁ represents the amplitude of a first input signal from one of the LPF circuits 102 and V₂ represents the amplitude of a second input signal from a different one of the LPF circuits 102. The x axis of the graph 600 shows the capacitance of the adjustable capacitor that receives V2 at its first terminal during calibration mode. In the example of FIG. 6, the signal 602 represents the relationship between the x and y axis of the graph 600 for when the adjustable capacitors 402 have a range of approximately 128 picofarads (pF) and a resolution of approximately 2 pF. In other examples, one or more of the adjustable capacitors 402 may have different ranges or different resolutions.

In general, two signals have the same phase if the difference between their amplitudes, when measured at the same time, is zero. Thus, the calibration circuitry 426 changes the phase of the second input signal to match that of the first input signal by finding the capacitance value that minimizes the y value in the signal 602.

The graph 600 shows that the signal 602 is non-linear. For example, suppose that the difference in phase between the first and second input signals is a positive value at C=0 pF. In such examples, increasing the value of the adjustable capacitor from 0 pF decreases the magnitude of the phase shift for a period. However, because the signal 602 is non-linear, it is possible to overshoot the ideal capacitance value that minimizes (V₁− V₂). Thus, at C=128 pF in the same example, the phase shift between the first and second input signals is a negative value instead of a positive one.

In view of the non-linear relationship between differential amplitude and capacitance shown in the signal 602, the calibration circuitry 426 implements a modified binary search to identify the capacitance value that minimizes (V₁−V₂) with the fewest number of comparisons.

In the example of FIG. 4, an adjustable capacitor 402A is implemented by n capacitors that can connect or disconnect to one another in parallel based on instructions from the calibration circuitry 426. Moreover, the n capacitors have different values so that the adjustable capacitor as a whole has an n-bit resolution. Thus, the total capacitance of the adjustable capacitor value 402A is an n-bit binary number where the most significant bit (MSB) is ‘1’ when a first capacitor is coupled to the other capacitors in parallel, and is ‘0’ when the first capacitor is decoupled to the other capacitors. Similarly, the value of the next most significant bit is determined by a second capacitor that has half the capacitance as the first capacitor, etc.

To identify the overall capacitance value of an adjustable capacitor 402A that minimizes (V₁− V₂) with the fewest number of comparisons, the calibration circuitry 426 measures the differential amplitude at two different test capacitance values that are equidistant from a midpoint capacitance value. The calibration circuitry 426 then compares the two measurements to determine which is smaller. The test value that corresponds to the smaller of the two measurements then becomes the midpoint value for the next iteration, and the calibration circuitry 426 repeats the foregoing analysis. The modified binary search ends when the differential amplitude at a modified midpoint value is smaller than either of the differential amplitudes at the next two test values. Because a given adjustable capacitor implements n capacitors that can couple to one another in parallel, a single iteration of the modified binary search sets the value of one bit in the n-bit binary number and determines whether the corresponding capacitor is coupled or decoupled from the others.

The following example utilizes an adjustable capacitor that has a range of values shown in FIG. 6 (e.g., 0 to 128 pF), a 2 pF resolution, and has n=6 as described above. In such an example, a first iteration of the modified binary search has a midpoint value of 64 pF, a first test value of 32 pF, and a second test value of 96 pF. The calibration circuitry 426 measures the differential amplitude at the two test values and determines the value of (V₁− V₂) is smaller at C=96 pF than the value of (V₁− V₂) at C=32 pF (as shown in the graph 600). Accordingly, the MSB of the 6-bit adjustable capacitor is set to ‘1’ (e.g. 1xxxxx, where ‘x’ represents a currently unknown bit), and the corresponding capacitor is coupled to the others in parallel, because the overall capacitance value that minimizes (V₁− V₂) is greater or equal to 64 pF.

In a second iteration of the modified binary search shown in the example of FIG. 6, the midpoint value is 96 pF, the first test value is 80 pF, and the second test value is 112 pF. The calibration circuitry 426 measures the differential amplitude at the two test values and determines the value of (V₁− V₂) is smaller at C=80 pF than the value of (V₁− V₂) at C=112 pF (as shown in the graph 600). Accordingly, the next most significant bit of the 6-bit adjustable capacitor is set to ‘0’ (e.g., 10xxxx), and the corresponding capacitor is not coupled to the others in parallel, because the overall capacitance value that minimizes (V₁− V₂) is less than [(1)64 pF+(1)32 pF=96 pF].

In a third iteration of the modified binary search shown in the example of FIG. 6, the midpoint value is 80 pF, the first test value is 72 pF, and the second test value is 88 pF. The calibration circuitry 426 measures the differential amplitude at the two test values and determines the value of (V₁− V₂) is smaller at C=88 pF than the value of (V₁− V₂) at C=72 pF. Accordingly, the next most significant bit of the 6-bit adjustable capacitor is set to ‘1’ (e.g., 101xxx), and the corresponding capacitor is coupled to the others in parallel, because the overall capacitance value that minimizes (V₁− V₂) is greater than or equal to [(1)64 pF+(0)32 pF+(1)16 pF=80 pF].

In a fourth iteration of the modified binary search shown in the example of FIG. 6, the midpoint value is 88 pF, the first test value is 84 pF, and the second test value is 92 pF. The calibration circuitry 426 measures the differential amplitude at the two test values and determines the value of (V₁− V₂) is smaller at C=84 pF than the value of (V₁− V₂) at C=92 pF. Accordingly, the next most significant bit of the 6-bit adjustable capacitor is set to ‘0’ (e.g., 1010 xx), and the corresponding capacitor is not coupled to the others in parallel, because the overall capacitance value that minimizes (V₁− V₂) is less than [(1)64 pF+(0)32 pF+(1)16 pF+(1)8 pF=88 pF].

In a fifth iteration of the modified binary search shown in the example of FIG. 6, the midpoint value is 84 pF, the first test value is 82 pF, and the second test value is 86 pF. Here, the calibration circuitry 426 measures the differential amplitude at the two test values and determines the value of (V₁− V₂) is smaller at C=86 pF than the value of (V₁− V₂) at C=82 pF. Accordingly, the next most significant bit of the 6-bit adjustable capacitor is set to ‘1’ (e.g., 10101x), and the corresponding capacitor is coupled to the others in parallel, because the overall capacitance value that minimizes (V₁− V₂) is greater than or equal to [(1)64 pF+(0)32 pF+(1)16 pF+(0)8 pF+(1)4 pF=84 pF].

In the last iteration of the modified binary search, the midpoint value is 86 pF, the first test value is 84 pF, and the second test value is 88 pF. Because the binary search is at its last iteration, the calibration circuitry 426 measures the differential amplitudes at three values rather than two: first test value, second test value, and midpoint value. In this example, the calibration circuitry 426 determines the value of (V₁− V₂) at the first test value, 84 pF, is smaller than the value of (V₁− V₂) at either of the second test value or the midpoint value. Accordingly, the least significant bit (LSB) of the 6-bit adjustable capacitor is set to ‘0’ (e.g., 101010), and the corresponding capacitor is not coupled to the others in parallel, because the overall capacitance value that minimizes (V₁− V₂) is [(1)64 pF+(0)32 pF+(1)16 pF+(0)8 pF+(1)4 pF=84 pF].

At the end of the first instance of the modified binary search, the calibration circuitry 426 has adjusted one of the adjustable capacitors 402 and matched the phases of two input signals. The calibration circuitry 426 then implements another instance of the modified binary search on a different one of the adjustable capacitors 402, thereby changing the value of V2 described above. Notably, the selected input signal described above as V₁, and the value of its corresponding adjustable capacitor do not change throughout any instance of the modified binary searches.

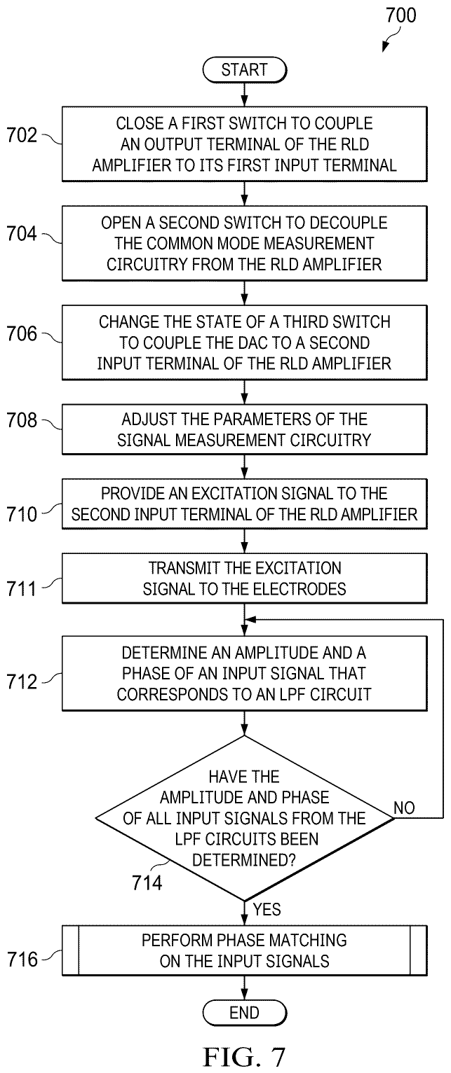

FIG. 7 is a flowchart representative of example machine-readable instructions or example operations 700 that may be at least one of executed, instantiated, or performed using or more of the front-end circuitry 104 and the controller circuitry 112 of FIG. 1 to implement calibration mode. In the example flowchart of FIG. 7, calibration mode begins when the controller circuitry 112 closes a first switch to couple an output terminal of an RLD amplifier circuit to one its first input terminal. (Block 702). In the example of FIG. 5, the first switch referred to in block 702 is the switch 502 and the first input terminal referred to in block 702 is the negative terminal of the RLD amplifier circuitry 510. The closed switch at block 702 enables the RLD amplifier circuitry 510 to function as a buffer and decouples the compensation circuitry 110 during calibration mode.

The controller circuitry 112 opens a second switch to decouple common mode measurement circuitry 508 from the RLD amplifier circuitry 510. (Block 704). In this example, the second switch of block 704 refers to switch 504 as shown in FIG. 5. Switch 504 is opened at block 704 because a common mode signal is not transmitted, and the common mode measurement circuitry 508 is therefore not utilized, during calibration mode.

The controller circuitry 112 changes the state of a third switch to couple the DAC circuitry 518 to a second input terminal of the RLD amplifier circuitry 510. (Block 706). In this example, the third switch of block 706 refers to switch 506 as shown in FIG. 5. During calibration mode, the switch 506 changes from a second terminal that receives an expected common mode signal to a third terminal that is coupled to the DAC circuitry 518. The first terminal of the switch 506 remains coupled to the positive terminal of the RLD amplifier circuitry 510 during both calibration mode and measurement mode.

The controller circuitry 112 changes one or more parameters of the demodulation circuitry 424. (Block 708). The parameters used in block 708 refer to one or more values of x₁, y₁, zₗ, x₂, y₂, and z₂ as described in FIG. 5. The parameter values enable the demodulation circuitry 424 to accept the difference between V_{REF1} and an input signal, e.g., the output of the ADC circuitry 416, and measure the amplitude and phase of said input signal.

After the switches 502-506 are set to support calibration mode, the DDS circuitry 430 provides an excitation signal to the second input terminal of the RLD amplifier circuitry 510. (Block 710). The excitation signal is produced digitally by performing direct digital synthesis operations, converted into analog by the DAC circuitry 518, and reaches the RLD amplifier circuitry 510 because of the switch 506 that was set at block 706. In the example of FIG. 7, the excitation signal is a sine wave. In other examples, the excitation signal is a different type of waveform.

The RLD amplifier circuitry 510 transmits the excitation signal to the electrodes 118. (Block 711). The excitation signal travels through the RLD_OUT terminal of the front-end circuitry 104 shown in FIG. 5, through the RLD electrode 116, through the electrodes 118, and through the LPF circuits 102. Thus, modified versions of the excitation signal re-enter the front-end circuitry 104 as multiple input signals. The electrodes 118 are shorted together at block 711 to ensure the respective LPF circuits 102 receive the same version of the excitation signal. In some examples, the electrodes 118 are shorted together using a hardware interface on or coupled to the system board 100. In other examples, the electrodes 118 are shorted together by attaching the electrodes to the patient 114. The LPF circuits 102 modify the excitation signal at block 711 due to component mismatch.

The demodulation circuitry 424 determines an amplitude and a phase of an input signal that corresponds to one of the LPF circuits 102. (Block 712). The demodulation circuitry 424 uses the parameters set at block 708 to perform the measurements of block 712. The demodulation circuitry 424 then determines whether the amplitude and phase of all input signals that correspond to the LPF circuits 102 have been determined. (Block 714). If the amplitude and phase of one or more input signals have not yet been determined (Block 714: No), control returns to block 712 where the demodulation circuitry 424 analyzes another one of the remaining input signals.

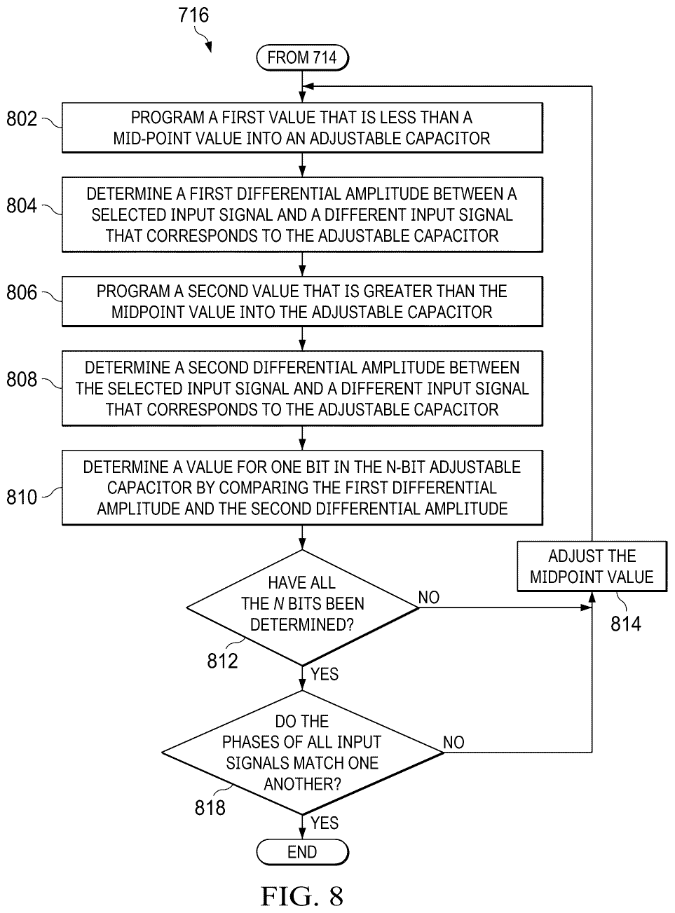

If the amplitude and phase of all input signals that correspond to the LPF circuits 102 have been determined (Block 714: Yes), the calibration circuitry 426 performs phase matching on the input signals of blocks 712 and 714. (Block 716). The phase matching operations of block 716 improve CMRR by ensuring that any subsequent phase shifts that occur during measurement mode are due to the performance of the organ in the patient 114, rather than component mismatch within the LPF circuits 102. Block 716 is described further in connection with FIG. 8.

FIG. 8 is a flowchart representative of example machine-readable instructions or example operations that may be at least one of executed, instantiated, or performed to match phases of input signals as described in FIG. 7. In particular, FIG. 8 is an example implementation of block 716 of FIG. 7.

Execution of block 716 begins when the calibration circuitry 426 programs a first value that is less than a midpoint value into one of the adjustable capacitors 402. (Block 802). In the example of FIG. 5, the midpoint value used at the first iteration of block 716 is the median value (64 pF) of the range of the adjustable capacitors (0-128 pF). In other examples, the midpoint value used at the first iteration of block 716 is a different value.

The front-end circuitry 104 determines a first differential amplitude between a selected input signal and a different input signal that corresponds to the adjustable capacitor. (Block 804). To do so, the DDS circuitry 430 and the driver circuitry 404 transmit an excitation signal to the electrodes 118 via the RLD electrode 116. In subsequent operations, the multiplexer 406 and switch 407 are configured to couple the selected input signal and a different input signal to the two inputs of INA circuitry 412. The demodulation circuitry 424 then determines the differential amplitude using the output of the INA circuitry 412.

In the example of FIG. 7, the selected input signal refers to a signal received from the LPF circuits 102 that has the largest phase value. In other examples, the selected input signal refers to a different one of the signals received from the LPF circuits 102.

The calibration circuitry 426 programs a second value that is greater than a midpoint value into the adjustable capacitor. (Block 806). In particular, the calibration circuitry 426 implements block 806 so that the difference between the second value and the midpoint is equal to the difference between the midpoint and the first value of block 802.

The front-end circuitry 104 determines a second differential amplitude between the selected input signal and a different input signal that corresponds to the adjustable capacitor. (Block 808). The front-end circuitry 104 implements block 808 using similar techniques to block 804.

The calibration circuitry 426 determines a value for one bit in the n-bit adjustable capacitor by comparing the first difference of block 804 to the second difference of block 806. (Block 810). The comparison of block 810 indicates which of the first difference or the second difference is smaller in magnitude. The calibration circuitry 426 then couples or decouples a corresponding capacitor to other capacitors in parallel responsive to the comparison. As described above in connection with FIG. 6, a bit is set to a logical ‘1’ and the total value of the adjustable capacitor increases when the corresponding capacitor of block 810 is coupled to the others parallel capacitors. Similarly, a bit is set to a logical ‘0’ and the total value of the adjustable capacitor does not increase when the corresponding capacitor of block 810 is decoupled to the other parallel capacitors.

The calibration circuitry 426 determines whether all n-bits in the adjustable capacitor of block 802 have been determined. (Block 812). If one or more bits of the adjustable capacitor have not yet been determined (Block 812: No), the calibration circuitry 426 adjusts the midpoint value. (Block 814). The midpoint value set at block 814 is the smaller of: a) the first value used at the previous iteration of block 802, or b) the second value used at the previous iteration of block 806. Control then returns to another iteration of block 802, where a new first value is programmed into the adjustable capacitor. In such examples where one or more bits of the adjustable capacitor have not yet been determined (Block 812: No), the adjustable capacitor used in the subsequent iteration of blocks 802-814 is the same adjustable capacitor used in the previous iteration of the loop.

If all n-bits in the adjustable capacitor of block 802 have been determined (Block 812: Yes), a single iteration of the modified binary search described has been completed. Accordingly, the calibration circuitry 426 then determines whether the phases of all the input signals received from the LPF circuits 102 match one another. (Block 818). If one or more input signals have a different phase from the other input signals (Block 818: No), control returns to block 814 where the calibration circuitry 426 resets the midpoint value. In examples where one or more input signals still require phase matching (Block 818: No), the adjustable capacitor referred to in the subsequent iteration of blocks 802-814 is a different one of the adjustable capacitors 402 from the previous iteration(s) of blocks 802-814. Alternatively, if all the input signals received from the LPF circuits 102 do match one another (Block 818: Yes), execution of both block 716 and calibration mode in general ends.

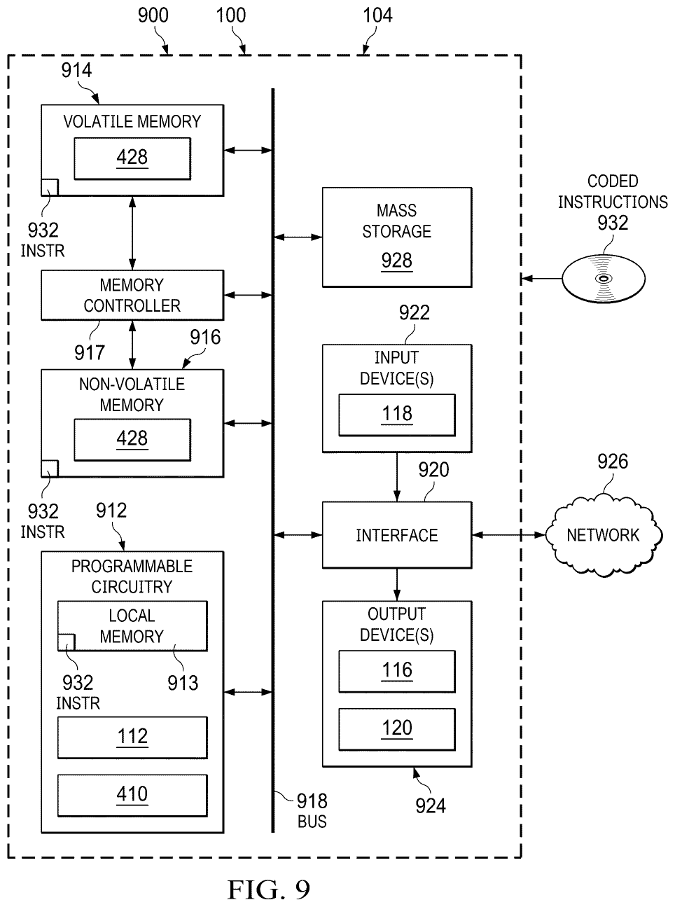

FIG. 9 is a block diagram of an example programmable circuitry platform 900 structured to one or a combination of execute or instantiate one or more of the example machine-readable instructions or the example operations of FIGS. 7 and 8 to implement the front-end cirucitry104, or, more generally, the system board 100 of FIG. 1. The programmable circuitry platform 900 can be, for example, a server, a personal computer, a workstation, a self-learning machine (e.g., a neural network), a mobile device (e.g., a cell phone, a smart phone, a tablet), an Internet appliance, a headset (e.g., an augmented reality (AR) headset, a virtual reality (VR) headset, etc.) or other wearable device, or any other type of computing or electronic device.

The programmable circuitry platform 900 of the illustrated example includes programmable circuitry 912. The programmable circuitry 912 of the illustrated example is hardware. For example, the programmable circuitry 912 can be implemented by one or more integrated circuits, logic circuits, FPGAs, microprocessors, CPUs, GPUs, DSPs, or microcontrollers from any desired family or manufacturer. The programmable circuitry 912 may be implemented by one or more semiconductor based (e.g., silicon based) devices. In this example, the programmable circuitry 912 implements the controller circuitry 112 and, within the front-end circuitry 104, the digital circuitry 410.

The programmable circuitry 912 of the illustrated example includes a local memory 913 (e.g., a cache, registers, etc.). The programmable circuitry 912 of the illustrated example is in communication with main memory 914, 916, which includes a volatile memory 914 and a non-volatile memory 916, by a bus 918. The volatile memory 914 may be implemented by one or more Synchronous Dynamic Random Access Memory (SDRAM), Dynamic Random Access Memory (DRAM), RAMBUS® Dynamic Random Access Memory (RDRAM®), or any other type of RAM device. The non-volatile memory 916 may be implemented by one or a combination of flash memory or any other desired type of memory device. Access to the main memory 914, 916 of the illustrated example is controlled by a memory controller 917. In some examples, the memory controller 917 may be implemented by one or more integrated circuits, logic circuits, microcontrollers from any desired family or manufacturer, or any other type of circuitry to manage the flow of data going to and from the main memory 914, 916. In this example, the main memory 914, 916 include the memory 428.

The programmable circuitry platform 900 of the illustrated example also includes interface circuitry 920. The interface circuitry 920 may be implemented by hardware in according to any type of interface standard, such as an Ethernet interface, a universal serial bus (USB) interface, a Bluetooth® interface, a near field communication (NFC) interface, a Peripheral Component Interconnect (PCI) interface, or a Peripheral Component Interconnect Express (PCIe) interface.

In the illustrated example, one or more input devices 922 are connected to the interface circuitry 920. The input device(s) 922 permit(s) a user (e.g., a human user, a machine user, etc.) to enter one of or a combination of data or commands into the programmable circuitry 912. The input device(s) 922 can be implemented by, for example, one of or a combination of an audio sensor, a microphone, a camera (still or video), a keyboard, a button, a mouse, a touchscreen, a trackpad, a trackball, an isopoint device, or a voice recognition system. In this example, the input device(s) 922 include the electrodes 118.

One or more output devices 924 are also connected to the interface circuitry 920 of the illustrated example. The output device(s) 924 can be implemented, for example, by one of or a combination of display devices (e.g., a light emitting diode (LED), an organic light emitting diode (OLED), a liquid crystal display (LCD), a cathode ray tube (CRT) display, an in-place switching (IPS) display, a touchscreen, etc.), a tactile output device, a printer, or speaker. The interface circuitry 920 of the illustrated example, thus, includes one of or a combination of a graphics driver card, a graphics driver chip, or graphics processor circuitry such as a GPU. In this example, the output device(s) 924 include the RLD electrode 116 and the display 120.

The interface circuitry 920 of the illustrated example also includes a communication device such as one of or a combination of a transmitter, a receiver, a transceiver, a modem, a residential gateway, a wireless access point, or a network interface to facilitate exchange of data with external machines (e.g., computing devices of any kind) by a network 926. The communication can be by, for example, an Ethernet connection, a digital subscriber line (DSL) connection, a telephone line connection, a coaxial cable system, a satellite system, a beyond-line-of-sight wireless system, a line-of-sight wireless system, a cellular telephone system, an optical connection, etc.

The programmable circuitry platform 900 of the illustrated example also includes one or more mass storage discs or devices 928 to store one or more of firmware, software, or data. Examples of such mass storage discs or devices 928 include one or more magnetic storage devices (e.g., floppy disk, drives, HDDs, etc.), optical storage devices (e.g., Blu-ray disks, CDs, DVDs, etc.), RAID systems, or solid-state storage discs or devices such as flash memory devices and SSDs.

The machine-readable instructions 932, which may be implemented by the machine-readable instructions of FIGS. 7 and 8, may be stored in one of or a combination of the mass storage device 928, in the volatile memory 914, in the non-volatile memory 916, or on at least one non-transitory computer readable storage medium such as a CD or DVD which may be removable.

While an example manner of implementing the system board 100 and front-end circuitry 104 is illustrated in FIGS. 1, 4, and 5, one or more of the elements, processes, or devices illustrated in FIG. 1 may be combined, divided, re-arranged, omitted, eliminated, or implemented in any other way. Further, the controller circuitry 112, the components within the digital circuitry 410, or, more generally, the example system board 100 and front-end circuitry 104 of FIGS. 1, 4, and 5, may be implemented by hardware alone or by hardware in combination with software and firmware. Thus, for example, any of the controller circuitry 112, the components within the digital circuitry 410, or, more generally, the example system board 100 and front-end circuitry 104 of FIGS. 1, 4, and 5, could be implemented by programmable circuitry in combination with one or more machine-readable instructions (e.g., firmware or software), processor circuitry, analog circuit(s), digital circuit(s), logic circuit(s), programmable processor(s), programmable microcontroller(s), graphics processing unit(s) (GPU(s)), digital signal processor(s) (DSP(s)), ASIC(s), programmable logic device(s) (PLD(s)), or field programmable logic device(s) (FPLD(s)) such as FPGAs. Further still, the example system board 100 and front-end circuitry 104 of FIGS. 1, 4, and 5 may include one or more elements, processes, or devices in addition to, or instead of, those illustrated in FIG. 1, or may include more than one of any or all of the illustrated elements, processes and devices.

Flowchart(s) representative of example machine-readable instructions, which may be executed by programmable circuitry to at least one of implement or instantiate the system board 100 and front-end circuitry 104 of FIGS. 1, 4, and 5 or representative of example operations which may be performed by programmable circuitry to at least one of implement or instantiate the system board 100 and front-end circuitry 104 of FIGS. 1, 4, and 5, are shown in FIGS. 7 and 8. The machine-readable instructions may be one or more executable programs or portion(s) of one or more executable programs for execution by programmable circuitry such as the programmable circuitry 912 shown in the example programable circuitry platform 900 described below in connection with FIG. 9 and may be one or more function(s) or portion(s) of functions to be performed by the example programmable circuitry (e.g., an FPGA). In some examples, the machine-readable instructions control an operation, a task, etc., to be carried out or performed in an automated manner in the real-world. As used herein, “automated” means without human involvement.

The program may be embodied in instructions (e.g., software and/or firmware) stored on one or more non-transitory computer readable and/or machine-readable storage medium such as one of or a combination of cache memory, a magnetic-storage device or disk (e.g., a floppy disk, a Hard Disk Drive (HDD), etc.), an optical-storage device or disk (e.g., a Blu-ray disk, a Compact Disk (CD), a Digital Versatile Disk (DVD), etc.), a Redundant Array of Independent Disks (RAID), a register, ROM, a solid-state drive (SSD), SSD memory, non-volatile memory (e.g., electrically erasable programmable read-only memory (EEPROM), flash memory, etc.), volatile memory (e.g., Random Access Memory (RAM) of any type, etc.), or any other storage device or storage disk. The instructions of the non-transitory computer readable and/or machine-readable medium may program or be executed by programmable circuitry located in one or more hardware devices, but the entire program or parts thereof could alternatively be executed or instantiated by one or more hardware devices other than the programmable circuitry or embodied in dedicated hardware. The machine-readable instructions may be distributed across multiple hardware devices or executed by two or more hardware devices (e.g., a server and a client hardware device). For example, the client hardware device may be implemented by an endpoint client hardware device (e.g., a hardware device associated with a human and/or machine user) or an intermediate client hardware device gateway (e.g., a radio access network (RAN)) that may facilitate communication between a server and an endpoint client hardware device. Similarly, the non-transitory computer readable storage medium may include one or more mediums. Further, although the example program is described with reference to the flowchart(s) illustrated in FIGS. 7 and 8, many other methods of implementing the example front-end circuitry 104 may alternatively be used. For example, the order of execution of the blocks of the flowchart(s) may be changed, or some of the blocks described may be changed, eliminated, or combined. Also or alternatively, any or all of the blocks of the flow chart may be implemented by one or more hardware circuits (e.g., processor circuitry, discrete, integrated analog and/or digital circuitry, an FPGA, an ASIC, a comparator, an operational-amplifier (op-amp), a logic circuit, etc.) structured to perform the corresponding operation without executing software or firmware. The programmable circuitry may be distributed in different network locations or local to one or more hardware devices (e.g., a single-core processor (e.g., a single core CPU), a multi-core processor (e.g., a multi-core CPU, an XPU, etc.)). For example, the programmable circuitry may be one of or a combination of a CPU or an FPGA located in the same package (e.g., the same integrated circuit (IC) package or in two or more separate housings), one or more processors in a single machine, multiple processors distributed across multiple servers of a server rack, multiple processors distributed across one or more server racks, etc., or any combination(s) thereof.

The machine-readable instructions described herein may be stored in one or more of a compressed format, an encrypted format, a fragmented format, a compiled format, an executable format, a packaged format, etc. Machine readable instructions as described herein may be stored as data (e.g., computer-readable data, machine-readable data, one or more bits (e.g., one or more computer-readable bits, one or more machine-readable bits, etc.), a bitstream (e.g., a computer-readable bitstream, a machine-readable bitstream, etc.), etc.) or a data structure (e.g., as portion(s) of instructions, code, representations of code, etc.) that may be utilized to create, manufacture, or produce machine executable instructions. For example, the machine-readable instructions may be fragmented and stored on one or more storage devices, disks or computing devices (e.g., servers) located at the same or different locations of a network or collection of networks (e.g., in the cloud, in edge devices, etc.). The machine-readable instructions may require one or more of installation, modification, adaptation, updating, combining, supplementing, configuring, decryption, decompression, unpacking, distribution, reassignment, compilation, etc., for them to be directly readable, interpretable, or executable by a computing device or other machine. For example, the machine-readable instructions may be stored in multiple parts, which are individually compressed, encrypted, or stored on separate computing devices, so that the parts when decrypted, decompressed, or combined form a set of one or more computer-executable or machine executable instructions that implement one or more functions or operations that may together form a program such as that described herein.

In another example, the machine-readable instructions may be stored in a state in which they may be read by programmable circuitry, but require addition of a library (e.g., a dynamic link library (DLL)), a software development kit (SDK), an application programming interface (API), etc., in order to execute the machine-readable instructions on a particular computing device or other device. In another example, the machine-readable instructions may need to be configured (e.g., settings stored, data input, network addresses recorded, etc.) before the machine-readable instructions or the corresponding program(s) can be executed in whole or in part. Thus, machine-readable, computer readable or machine-readable media, as used herein, may include one or a combination of instructions and program(s) regardless of the particular format or state of the machine-readable instructions or program(s).

The machine-readable instructions described herein can be represented by any past, present, or future instruction language, scripting language, programming language, etc. For example, the machine-readable instructions may be represented using any of the following languages: C, C++, Java, C#, Perl, Python, JavaScript, HyperText Markup Language (HTML), Structured Query Language (SQL), Swift, etc.

As mentioned above, the example operations of FIGS. 7 and 8 may be implemented using executable instructions (e.g., computer readable and/or machine-readable instructions) stored on one or more non-transitory computer readable or machine-readable media. As used herein, the terms non-transitory computer readable medium, non-transitory computer readable storage medium, non-transitory machine-readable medium, and non-transitory machine-readable storage medium are expressly defined to include any type of computer readable storage device or storage disk and to exclude propagating signals and to exclude transmission media. Examples of such non-transitory computer readable medium, non-transitory computer readable storage medium, non-transitory machine-readable medium, or non-transitory machine-readable storage medium include one or more optical storage devices, magnetic storage devices, an HDD, a flash memory, a read-only memory (ROM), a CD, a DVD, a cache, a RAM of any type, a register, or any other storage device or storage disk in which information is stored for any duration (e.g., for extended time periods, permanently, for brief instances, for temporarily buffering, for caching of the information). As used herein, the terms “non-transitory computer readable storage device” and “non-transitory machine-readable storage device” are defined to include any physical (mechanical, magnetic, electromechanical, or electrical) hardware to retain information for a time period, but to exclude propagating signals and to exclude transmission media. Examples of non-transitory computer readable storage devices or non-transitory machine-readable storage devices include one or a combination of random-access memory of any type, read only memory of any type, solid state memory, flash memory, optical discs, magnetic disks, disk drives, or redundant array of independent disks (RAID) systems. As used herein, the term “device” refers to physical structure such as one of or a combination of mechanical, electromechanical, or electrical equipment, hardware, or circuitry that may or may not be configured by computer readable instructions, machine-readable instructions, etc., or manufactured to execute computer-readable instructions, machine-readable instructions, etc.

“Including” and “comprising” (and all forms and tenses thereof) are used herein to be open ended terms. Thus, whenever a claim employs any form of “include” or “comprise” (e.g., comprises, includes, comprising, including, having, etc.) as a preamble or within a claim recitation of any kind, additional elements, terms, etc., may be present without falling outside the scope of the corresponding claim or recitation. As used herein, when the phrase “at least” is used as the transition term in, for example, a preamble of a claim, it is open-ended in the same manner as the term “comprising” and “including” are open ended. The term “and/or” when used, for example, in a form such as A, B, and/or C refers to any combination or subset of A, B, C such as (1) A alone, (2) B alone, (3) C alone, (4) A with B, (5) A with C, (6) B with C, or (7) A with B and with C. As used herein in the context of describing structures, components, items, objects and things, the phrase “at least one of A and B” refers to implementations including any of (1) at least one A, (2) at least one B, or (3) at least one A and at least one B. Similarly, as used herein in the context of describing structures, components, items, objects and things, the phrase “at least one of A or B” refers to implementations including any of (1) at least one A, (2) at least one B, or (3) at least one A and at least one B. As used herein in the context of describing the performance or execution of processes, instructions, actions, activities, etc., the phrase “at least one of A and B” refers to implementations including any of (1) at least one A, (2) at least one B, or (3) at least one A and at least one B. Similarly, as used herein in the context of describing the performance or execution of processes, instructions, actions, activities, etc., the phrase “at least one of A or B” refers to implementations including any of (1) at least one A, (2) at least one B, or (3) at least one A and at least one B.

As used herein, singular references (e.g., “a,” “an,” “first,” “second,” etc.) do not exclude a plurality. The term “a” or “an” object, as used herein, refers to one or more of that object. The terms “a” (or “an”), “one or more,” and “at least one” are used interchangeably herein. Furthermore, although individually listed, a plurality of means, elements, or actions may be implemented by, e.g., the same entity or object. Also, although individual features may be included in different examples or claims, these may possibly be combined, and the inclusion in different examples or claims does not imply that a combination of features is at least one of not feasible or advantageous.

As used herein, unless otherwise stated, the term “above” describes the relationship of two parts relative to Earth. A first part is above a second part, if the second part has at least one part between Earth and the first part. Likewise, as used herein, a first part is “below” a second part when the first part is closer to the Earth than the second part. As noted above, a first part can be above or below a second part with one or more of: other parts therebetween, without other parts therebetween, with the first and second parts touching, or without the first and second parts being in direct contact with one another.

Unless specifically stated otherwise, descriptors such as “first,” “second,” “third,” etc., are used herein without imputing or otherwise indicating any meaning of priority, physical order, arrangement in a list, or ordering in any way, but are merely used as at least one of labels or arbitrary names to distinguish elements for ease of understanding the described examples. In some examples, the descriptor “first” may be used to refer to an element in the detailed description, while the same element may be referred to in a claim with a different descriptor such as “second” or “third.” In such instances, such descriptors are used merely for identifying those elements distinctly within the context of the discussion (e.g., within a claim) in which the elements might, for example, otherwise share a same name.

As used herein, “approximately” and “about” modify their subjects/values to recognize the potential presence of variations that occur in real world applications. For example, “approximately” and “about” may modify dimensions that may not be exact due to at least one of manufacturing tolerances or other real-world imperfections. For example, “approximately” and “about” may indicate such dimensions may be within a tolerance range of +/−10% unless otherwise specified herein.

As used herein, the phrase “in communication,” including variations thereof, encompasses one of or a combination of direct communication or indirect communication through one or more intermediary components, and does not require direct physical (e.g., wired) communication or constant communication, but rather also includes selective communication at least one of periodic intervals, scheduled intervals, aperiodic intervals, or one-time events.

As used herein, “programmable circuitry” is defined to include at least one of (i) one or more special purpose electrical circuits (e.g., an application specific circuit (ASIC)) structured to perform specific operation(s) and including one or more semiconductor-based logic devices (e.g., electrical hardware implemented by one or more transistors), or (ii) one or more general purpose semiconductor-based electrical circuits programmable with instructions to perform one or more specific functions(s) or operation(s) and including one or more semiconductor-based logic devices (e.g., electrical hardware implemented by one or more transistors). Examples of programmable circuitry include programmable microprocessors such as Central Processor Units (CPUs) that may execute first instructions to perform one or more operations or functions, Field Programmable Gate Arrays (FPGAs) that may be programmed with second instructions to at least one of configure or structure the FPGAs to instantiate one or more operations or functions corresponding to the first instructions, Graphics Processor Units (GPUs) that may execute first instructions to perform one or more operations or functions, Digital Signal Processors (DSPs) that may execute first instructions to perform one or more operations or functions, XPUs, Network Processing Units (NPUs) one or more microcontrollers that may execute first instructions to perform one or more operations or functions or integrated circuits such as Application Specific Integrated Circuits (ASICs). For example, an XPU may be implemented by a heterogeneous computing system including multiple types of programmable circuitry (e.g., one or more FPGAs, one or more CPUs, one or more GPUs, one or more NPUs, one or more DSPs, etc., and any combination(s) thereof), and orchestration technology (e.g., application programming interface(s) (API(s)) that may assign computing task(s) to whichever one(s) of the multiple types of programmable circuitry is/are suited and available to perform the computing task(s).

In this description, the term “couple” may cover connections, communications, or signal paths that enable a functional relationship consistent with this description. For example, if device A generates a signal to control device B to perform an action: (a) in a first example, device A is coupled to device B by direct connection; or (b) in a second example, device A is coupled to device B through intervening component C if intervening component C does not alter the functional relationship between device A and device B, such that device B is controlled by device A via the control signal generated by device A.

A device that is “configured to” perform a task or function may be configured (e.g., at least one of programmed or hardwired) at a time of manufacturing by a manufacturer to at least one of perform the function or be configurable (or re-configurable) by a user after manufacturing to perform the function/or other additional or alternative functions. The configuring may be through at least one of firmware or software programming of the device, through at least one of a construction or layout of hardware components and interconnections of the device, or a combination thereof.

As used herein, the terms “terminal,” “node,” “interconnection,” “pin” and “lead” are used interchangeably. Unless specifically stated to the contrary, these terms are generally used to mean an interconnection between or a terminus of a device element, a circuit element, an integrated circuit, a device or other electronics or semiconductor component.

In the description and claims, described “circuitry” may include one or more circuits. A circuit or device that is described herein as including certain components may instead be adapted to be coupled to those components to form the described circuitry or device. For example, a structure described as including one or more semiconductor elements (such as transistors), one or more passive elements (such as one of or a combination of resistors, capacitors, or inductors), or one or more sources (such as voltage and/or current sources) may instead include only the semiconductor elements within a single physical device (e.g., at least one of a semiconductor die or integrated circuit (IC) package) and may be adapted to be coupled to at least some of the passive elements or the sources to form the described structure either at a time of manufacture or after a time of manufacture, for example, by at least one of an end-user or a third-party.

Circuits described herein are reconfigurable to include the replaced components to provide functionality at least partially similar to functionality available prior to the component replacement. Components shown as resistors, unless otherwise stated, are generally representative of any one or more elements coupled in at least one of series or parallel to provide an amount of impedance represented by the shown resistor. For example, a resistor or capacitor shown and described herein as a single component may instead be multiple resistors or capacitors, respectively, coupled in parallel between the same nodes. For example, a resistor or capacitor shown and described herein as a single component may instead be multiple resistors or capacitors, respectively, coupled in series between the same two nodes as the single resistor or capacitor. While certain elements of the described examples are included in an integrated circuit and other elements are external to the integrated circuit, in other example embodiments, additional or fewer features may be incorporated into the integrated circuit. In addition, some or all of the features illustrated as being external to the integrated circuit may be included in the integrated circuit and some features illustrated as being internal to the integrated circuit may be incorporated outside of the integrated. As used herein, the term “integrated circuit” means one or more circuits that are at least one of: (i) incorporated in/over a semiconductor substrate; (ii) incorporated in a single semiconductor package; (iii) incorporated into the same module; or (iv) incorporated in/on the same printed circuit board.

Uses of the phrase “ground” in the foregoing description include at least one of a chassis ground, an Earth ground, a floating ground, a virtual ground, a digital ground, a common ground, or any other form of ground connection applicable to, or suitable for, the teachings of this description.

Modifications are possible in the described embodiments, and other embodiments are possible, within the scope of the claims.

From the foregoing, it will be appreciated that example systems, apparatus, articles of manufacture, and methods have been described that calibrate electrocardiogram systems in a manner that is simpler, consumes less power, can be implemented with less space, and costs less than other approaches. Described systems, apparatus, articles of manufacture, and methods improve the efficiency of using a computing device by changing the value of one or more adjustable capacitors so that the phase of input signals received during calibration mode match one another. The matched phase signals improve CMRR because they remove the possibility that subsequent phase shifts that are observed during a measurement mode are attributable to component mismatch within LPF circuits. The front-end circuitry 104 described in examples herein uses the adjustable capacitors to match phases while including only one analog signal chain, differential signal processing, and a clock signal with a comparatively low frequency. Described systems, apparatus, articles of manufacture, and methods are also directed to one or more improvement(s) in the operation of a machine such as a computer or other electronic, electromechanical, or mechanical device.

What is claimed is:

1. An apparatus comprising:
an adjustable capacitor having a first terminal coupled to an input terminal, a second terminal coupled to ground, and a control terminal;
demodulation circuitry having an input coupled to the first terminal of the adjustable capacitor;
calibration circuitry having an input coupled to an output of the demodulation circuitry and an output coupled to the control terminal of the adjustable capacitor; and
driver circuitry having an input coupled to the adjustable capacitor and an output coupled to an output terminal.

2. The apparatus of claim 1, wherein the driver circuitry includes:
right leg drive (RLD) amplifier circuitry having a positive terminal, a negative terminal, and an output;
a first switch having a first terminal coupled to the output of the RLD amplifier circuitry, a second terminal coupled to the negative terminal of the RLD amplifier circuitry, and a control terminal;
a second switch having a first terminal coupled to the negative terminal of the RLD amplifier circuitry, a second terminal, and a control terminal;
common mode measurement circuitry having an input coupled to the first terminal of the adjustable capacitor, the common mode measurement circuitry also having an output coupled to the second terminal of the second switch;
a third switch having a first terminal coupled to the positive terminal of the RLD amplifier circuitry, a second terminal, a third terminal, and a control terminal; and
Digital to Analog Converter (DAC) circuitry having an output coupled to the second terminal of the third switch.

3. The apparatus of claim 2, further including current-to-voltage converter circuitry having a first terminal coupled to the output of the DAC circuitry and a second terminal coupled to the second terminal of the second switch.

4. The apparatus of claim 2, further including Direct Digital Synthesis (DDS) circuitry having an output coupled to the DAC circuitry.

5. The apparatus of claim 1, wherein the demodulation circuitry includes:
multiplier circuitry having an input coupled to the first terminal of the adjustable capacitor and a second input;
first decimation circuitry having an input coupled to an output of the multiplier circuitry;
gain circuitry having an input coupled an output of the first decimation circuitry;
coordinate rotation digital computer (CORDIC) circuitry having an input coupled to an output of the gain circuitry; and
second decimation circuitry having an input coupled to an output of the CORDIC circuitry and an output coupled to the input of the calibration circuitry.

6. The apparatus of claim 5, wherein the demodulation circuitry further includes:
second multiplier circuitry having an input coupled to the first terminal of the adjustable capacitor and a second input;
third decimation circuitry having an input coupled to an output of the second multiplier circuitry;
second gain circuitry having an input coupled to the third decimation circuitry and an output coupled to the CORDIC circuitry; and
fourth decimation circuitry having an input coupled to an output of the CORDIC circuitry and an output coupled to the input of the calibration circuitry.

7. The apparatus of claim 1, wherein
the adjustable capacitor is a first adjustable capacitor;
the input terminal is a first input terminal; and
the apparatus further includes:
a second input terminal; and
a second adjustable capacitor having a first terminal coupled to the second input terminal, a second terminal coupled to ground, and a control terminal.

8. The apparatus of claim 7, further including:
a multiplexer having a first input coupled to the first terminal of the first adjustable capacitor, a second input coupled to the first input of the second adjustable capacitor, and an output;
instrumentation amplifier circuitry having an input coupled to the output of the multiplexer and an output;
anti-aliasing filter (AAF) circuitry having an input coupled to the output of the instrumentation amplifier circuitry and an output; and
Analog-to-Digital Conversion (ADC) circuitry having an input coupled to the output of the AAF circuitry and an output coupled to the demodulation circuitry.

9. An apparatus comprising:
a first electrode, a second electrode, and a third electrode;
first Low Pass Filter (LPF) circuitry having an input coupled to the first electrode and an output;
second LPF circuitry having an input coupled to the second electrode and an output; and
front-end circuitry having:
a first input terminal coupled to the output of the first LPF circuitry;
a first adjustable capacitor coupled to the first input terminal;
a second input terminal coupled to the output of the second LPF circuitry;
a first output terminal coupled to the third electrode; and
a second output terminal coupled to controller circuitry.

10. The apparatus of claim 9, wherein the first LPF circuitry includes:
a first resistor having a first terminal coupled to the first electrode and a second terminal;
a first capacitor having a first terminal coupled to the second terminal of the first resistor and a second terminal coupled to ground;
a second resistor having a first terminal coupled to the second terminal of the first resistor and to the first adjustable capacitor; and
a second capacitor having a first terminal coupled to the second terminal of the second resistor and a second terminal coupled to ground.

11. The apparatus of claim 10, wherein the second LPF circuitry includes:
a first resistor having a first terminal coupled to the second electrode and a second terminal;
a first capacitor having a first terminal coupled to the second terminal of the first resistor and a second terminal coupled to ground;
a second resistor having a first terminal coupled to the second terminal of the first resistor and to the first adjustable capacitor; and
a second capacitor having a first terminal coupled to the second terminal of the second resistor and a second terminal coupled to ground.

12. The apparatus of claim 11, wherein a magnitude of a differential signal analyzed by the front-end circuitry is proportional to:
a difference in resistance values between the first resistor of the first LPF circuitry and the first resistor of the second LPF circuitry;
a difference in resistance values between the second resistor of the first LPF circuitry and the second resistor of the second LPF circuitry;
a difference in capacitance values between the first capacitor of the first LPF circuitry and the first capacitor of the second LPF circuitry; or
a difference in capacitance values between the second capacitor of the first LPF circuitry and the second capacitor of the second LPF circuitry.

13. The apparatus of claim 12, wherein the front-end circuitry is configured to increase a common mode rejection ratio of the differential signal by changing a capacitance value of the first adjustable capacitor.

14. The apparatus of claim 13, wherein the front-end circuitry is configured to operate in an electro-surgical interference (ESI) operations after changing the capacitance value of the first adjustable capacitor.

15. A method comprising:
transmitting an excitation signal at an output terminal of front-end circuitry;
determining an amplitude and phase of a first input signal received at a first input terminal of the front-end circuitry, wherein the first input signal corresponds to first low pass filter (LPF) circuitry;
determining an amplitude and phase of a second input signal received at a second input terminal of the front-end circuitry, wherein the second input signal corresponds to a second LPF circuitry; and
matching the phase of the second input signal to the phase of the first input signal by changing, responsive to a difference between the amplitudes, a capacitance of an adjustable capacitor coupled to the second input terminal within the front-end circuitry.

16. The method of claim 15, further including performing, with the front-end circuitry, direct digital synthesis operations to generate the excitation signal.

17. The method of claim 15, further including decoupling, with a switch and before transmitting the excitation signal, common mode measurement circuitry within the front-end circuitry from right leg drive amplifier circuitry within the front-end circuitry.

18. The method of claim 15, further including closing a switch within the front-end circuitry to decouple compensation circuitry from right leg drive amplifier circuitry within the front-end circuitry.

19. The method of claim 15, further including adjusting, before determining the phases, parameters of decimation circuitry, gain circuitry, and coordinate rotation digital computer (CORDIC) circuitry within the front-end circuitry.

20. The method of claim 15, wherein changing the capacitance of the adjustable capacitor includes:
determining a first differential amplitude between the first input signal and the second input signal, wherein the adjustable capacitor has a first capacitance value while an instrumentation amplifier within the front-end circuitry is receiving the first input signal and the second input signal;
determining a second differential amplitude between the first input signal and a third input signal, wherein the third input signal corresponds to the second LPF circuitry, wherein the adjustable capacitor has a second capacitance value while the instrumentation amplifier is receiving the first input signal and the third input signal, wherein the second capacitance value is different from the first capacitance value; and
comparing the first differential amplitude and the second differential amplitude.
